SEQUENCE LISTING (13 sequences)

SEQ ID NO: 1 - protein, 42 aa, Homo sapiens
DAEFRHDSGYEVHHQKLVFFAQDVGSNKGAIIGLMVGGVVIA

SEQ ID NO: 2 - DNA, 129 bp, Homo sapiens
gatgcagaattccgacatgactcaggatatgaagttcatcatcaaaaattggtgttctttgcacaagatgtgggttcaaacaaaggtgcaatcattggactcatggtgggcggtgttgtcatagcgtga

SEQ ID NO: 3 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - DNA, 1421 bp, Homo sapiens
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgccccc

SEQ ID NO: 5 - protein, 18 aa, Drosophila melanogaster
MDISYIFVICLMALSGGS

SEQ ID NO: 6 - protein, 24 aa, Drosophila melanogaster
MPTTLMLLPCMLLLLLTAAAVAVG

SEQ ID NO: 7 - protein, 27 aa, Drosophila melanogaster
MCAALRRNLLLRSLWVVLAIGTAQVQA

SEQ ID NO: 8 - protein, 20 aa, Drosophila melanogaster
MAAVNLQASCSSGLASEDDA

SEQ ID NO: 9 - protein, 23 aa, Drosophila melanogaster
MMHILVTLLLVAIHSIPTTWAVT

SEQ ID NO: 10 - DNA, 3747 bp, Homo sapiens
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaactccccactcctggtagctcagaccctctgatccaaccctccagccctgctgtgtgcccagagccaccttcctctcctaaacacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

SEQ ID NO: 11 - DNA, 2796 bp, Homo sapiens
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

SEQ ID NO: 12 - DNA, 2622 bp, Homo sapiens
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

SEQ ID NO: 13 - DNA, 2529 bp, Homo sapiens
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

(19) United States
(12) Patent Application Publication
(10) Pub. No.: US 2005/0132424 A1
(43) Pub. Date: Jun. 16, 2005

(54) Transgenic flies expressing Abeta42-Dutch

(75) Inventors: David A. Lowe, Boston, MA (US); Gerhard Koenig, Arlington, MA (US); Christopher J. Cummings, Brookline, MA (US)

Correspondence Address: PALMER & DODGE, LLP;KATHLEEN M. WILLIAMS, 111 HUNTINGTON AVENUE, BOSTON, MA, 02199, US

(73) Assignee: EnVivo Pharmaceuticals, Inc.

(21) Appl. No.: 10/969,898

(22) Filed: Oct. 21, 2004

Related U.S. Application Data
(63) Continuation-in-part of application No. 10/853,593, filed on May 25, 2004.
(60) Provisional application No. 60/512,972, filed on Oct. 21, 2003.

(51) Int. Cl. A01K 67/033

(52) U.S. Cl. 800/8; 800/9; 800/13

(57) ABSTRACT

The present invention discloses a transgenic fly that expresses the Dutch mutant version of the human Aβ42 peptide of human amyloid-β precursor protein (APP), and a double transgenic fly that expresses both the Tau protein and the human Aβ42_{Dutch} peptide of human amyloid-β precursor protein (APP). The transgenic flies of the present invention provide for models of neurodegenerative disorders, such as Alzheimer's disease. The invention further discloses methods for identifying genetic modifiers, as well as screening methods to identify therapeutic compounds to treat neurodegenerative disorders using the transgenic flies.

### RELATED APPLICATION

This application is a Continuation in part of U.S. application Ser. No. 10/853,593, filed May 25, 2004, which claims the benefit of U.S. Provisional Application No. 60/512,972, filed on Oct. 21, 2003. The entire teachings of the above applications are incorporated herein by reference.

### BACKGROUND

Alzheimer's disease (AD) is the most common neurodegenerative disorder in humans. The disease is characterized by a progressive impairment in cognition and memory. The hallmark of AD at the neuropathological level is the extracellular accumulation of the amyloid-p peptide (Aβ) in “senile” plaques, and the intracellular deposition of neurofibrillary tangles made of the microtubule-associated protein Tau. In neuronal tissue of AD patients, Tau is hyperphosphorylated and adopts pathological conformations evident with conformation-dependent antibodies. The amyloid-β peptide is a cleavage product of the amyloid precursor protein (APP). In normal individuals, most of Aβ is in a 40-amino acid form, but there are also minor amounts of Aβ that are 42 amino acids in length (Aβ42). In patients with AD, there is an overabundance of Aβ42 that is thought to be the main toxic Aβ form.

A number of pathogenic mutations have been found within APP which are associated with hereditary forms of AD, several of which are located within the Aβ sequences. These mutations result in a phenotype different from AD, with massive amyloid accumulation in cerebral blood vessel walls. Two mutations, nameiy the Dutch (Glu22Gln) and the Flemish (Ala21 Gly) mutations, have been reported (Levy, et al., Science 248, 1124-1126 (1990)), (van Broeckhoven et al. (1990)), (Hendriks, et al., Nature Genet 1, 218-221 (1992)). Patients having these mutations suffer from cerebral hemorrhage and vascular symptoms. The vascular symptoms are caused by aggregation of Aβ in blood vessel walls (amyloid angiopathy). A third pathogenic intra-Aβ; mutation was recently discovered in an Italian family (Glu22Lys), with clinical findings similar to the Dutch patients (Tagliavini, et al., Alz Report 2, S28 (1999)). Yet another pathogenic AD mutation within APP, named the “Arctic mutation” (Glu22Gly), is also located within the Aβ peptide domain of the APP gene. Carriers of this mutation develop progressive dementia with clinical features typical of AD without symptoms of cerebrovascular disease. AD is distinctly characterized by accelerated formation of protofibrils comprising mutated Aβ peptides (Aβ40_{ARC} and/or Aβ42_{ARC}) compared to protofibril formation of wild type Aβ peptides. Finally, carriers of the “Iowa” mutation, carrying a Asp23Asn mutation within Aβ, exhibit severe cerebral amyloid angiopathy, widespread neurofibrillary tangles, and unusually extensive distribution of Aβ40 in plaques. (Grabowski et al., Ann. Neurol. 49: 691-693 (2001)).

A number of transgenic mouse models have been generated that express wild-type or mutant human APP. The mutant form of APP is differentially cleaved to result in increased amounts of Aβ42 deposited within Aβ plaques. These transgenic mice present with neurological symptoms of Alzheimer's disease, such as impaired memory and motor function (Janus C. et al., Curr. Neurol. Neurosci. Rep 1 (5): 451-457 (2001)). A transgenic mouse that expresses both mutant human APP and mutant human Tau has also been generated (Jada, et. al., Science, (5534) 293:1487-1491 (2001)). This double transgenic mouse is a rodent model for AD that shows enhanced neurofibrillary degeneration indicating that either APP or Aβ influences the formation of neurofibrillary tangles.

Mouse models have proven very useful for testing potential AD therapeutics. However, the use of mice for testing therapeutics is both expensive and time consuming. Thus, it would be beneficial to find alternative models which are less expensive and that can be efficiently used to screen for therapeutic agents for Alzheimer's disease. For example, non-mammalian animal models, such as Caenorhabditis elegans or Drosophila melanogaster.

The use of Drosophila as a model organism has proven to be an important tool in the elucidation of human neurodegenerative pathways (reviewed in Fortini, M. and Bonini, N. Trends Genet. 16: 161-167 (2000)), as the Drosophila genome contains many relevant human orthologs that are extremely well conserved in function (Rubin, G. M., et al., Science 287: 2204-2215 (2000)). For example, Drosophila melanogaster carries a gene that is homologous to human APP which is involved in nervous system function. The gene, APP-like (Appl), is approximately 40% identical to the neurogenic isoform (Rosen et al., Proc. Natl. Acad. Sci. U.S.A. 86:2478-2482 (1988)) and, like human APP695, is exclusively expressed in the nervous system. Flies deficient for the Appl gene show behavioral defects which can be rescued by the human APP gene, suggesting that the two genes have similar functions in the two organisms (Luo et al., Neuron 9:595-605 (1992)).

In addition, Drosophila models of polyglutamine repeat diseases (Jackson, G. R., et al (1998). Neuron 21: 633-642; Kazemi-Esfarani, P. and Benzer, S. (2000). Science 287: 1837-1840; Femandez-Funez et al. (2000) Nature 408 (6808):101-6), Parkinson's disease (Feany, M. B. and Bender, W. W. (2000). Nature 404: 394-398) and others have been established which closely mimic the disease state in humans at the cellular and physiological levels, and have been successfully employed in identifying other genes that may be involved in these diseases. Thus, the power of Drosophila as a model system is demonstrated in the ability to represent the disease state and to perform large scale genetic screens to identify critical components of disease. This invention generally relates to a method to identify compounds and genes acting on the APP pathway in transgenic Drosophila melanogaster that ectopically express genes related to AD. Expression of these transgenes can induce visible phenotypes and it is contemplated herein that genetic screens disclosed herein may be used to identify genes involved in the APP pathway by the identification of mutations that modify the induced visible phenotypes. The genes affected by these mutations will be called herein “genetic modifiers”. It is contemplated herein that human homologs of such genetic modifiers would be useful targets in the development of therapeutics to treat conditions associated with, but not limited to, Alzheimer Disease.

### SUMMARY OF THE INVENTION

The present invention discloses transgenic flies that express the human Aβ42 peptide of APP containing the pathogenic ‘Dutch mutation’ (E22Q) within the Aβ42 (Aβ42_{Dutch}) peptide of SEQ ID NO: 1.

The present invention provides transgenic flies whose somatic and germ cells comprise a transgene encoding the human Aβ42_{Dutch} containing the Dutch mutation, and wherein expression of the transgene results in the fly having a predisposition to, or resulting in, progressive neural degeneration.

In one embodiment, the transgenic fly is transgenic Drosophila.

In a preferred embodiment of the invention, the transgenic fly comprises a second transgene, encoding the Tau protein. The double transgenic fly of this embodiment displays a synergistic altered phenotype as compared to the altered phenotype displayed by transgenic flies expressing mutant human Aβ42_{Dutch} alone.

In a more preferred embodiment of this invention, the Tau and human Aβ42_{Dutch} mutant transgenes are operatively linked to an expression control sequence and expression of the transgenes results in an observable phenotype. In one embodiment, the transgene is temporally regulated by the expression control sequence. In another embodiment, the transgene is spatially regulated by the expression control sequence. In a specific embodiment of the invention, the expression control sequence is a heat shock promoter. In a preferred mode of the embodiment, the heat shock promoter is derived from the hsp70 or hsp83 genes. In other specific embodiments, the Tau and human Aβ42_{Dutch} transgenes are operatively linked to a GAL4 Upstream Activating Sequence (“UAS”). Optionally, the transgenic Drosophila comprising Tau and human Aβ42_{Dutch} mutant transgenes further comprise a GAL4 gene. In a preferred embodiment, the GAL4 gene is linked to a tissue specific expression control sequence. In a preferred mode of the embodiment, the tissue specific expression control sequence is derived from the sevenless, eyeless, gmr/glass or any of the rhodopsin genes. In another preferred mode of the embodiment, the tissue specific expression control sequence is derived from the dpp, vestigial, or apterous genes. In another preferred mode of the embodiment, the tissue specific expression control sequence is derived from neural-specific genes like elav, nirvana or D42 genes. In yet other embodiments, the expression control sequence is derived from ubiquitously expressed genes like tubulin, actin, or ubiquitin. In yet other embodiments, the expression control sequence comprises a tetracycline-controlled transcriptional activator (tTA) responsive regulatory element. Optionally, the transgenic Drosophila comprising the Tau and mutant human Aβ42_{Dutch} transgenes further comprise a tTA gene.

The DNA sequence encoding the mutant human Aβ42_{Dutch} may be fused to a signal peptide, e.g., via an amino acid linker. The signal peptide may be a wingless (wg) signal peptide, such as the peptide represented by SEQ ID NO: 5, or an Argos (aos) signal peptide, such as the sequence of SEQ ID NO: 6. The transgenic fly may exhibit an altered phenotype, such as a rough eye phenotype, a concave wing phenotype, a locomotor dysfunction (e.g., reduced climbing ability, reduced walking ability, reduced flying ability, decreased speed, abnormal trajectories, and abnormal turnings), abnormal grooming, other abnormal behaviors, or reduced life span.

In another aspect, the invention relates to a method for identifying an agent active in neurodegenerative disease. The method comprises the steps of: (a) providing a transgenic fly whose genome comprises DNA sequences that encode the mutant human Aβ42_{Dutch} alone, or in combination with the Tau protein; (b) providing a candidate agent to the transgenic fly; and (c) observing the phenotype of the transgenic fly of step (b) relative to the control fly that has not been administered an agent. An observable difference in the phenotype of the transgenic fly that has been administered an agent compared to the control fly that has not been administered an agent is indicative of an agent active in neurodegenerative disease. In yet another aspect, the invention relates to a method for identifying an agent active in neurodegenerative disease. The method comprises the steps of: (a) providing a transgenic fly and a control wild-type fly; (b) providing a candidate agent to the transgenic fly and to the control fly; and (c) observing a difference in phenotype between the transgenic fly and the control fly, wherein a difference in phenotype is indicative of an agent active in neurodegenerative disease.

In a further aspect, the invention relates to a method to identify genetic modifiers of the APP pathway, comprising: providing a transgenic fly whose genome comprises a DNA sequence encoding a polypeptide comprising the Aβ42_{DUTCH} (SEQ. ID NO:2) which is optionally fused to a signal sequence, alone or together with DNA sequence encoding the Tau, where the DNA sequence is operably linked to a tissue-specific expression control sequence; and wherein expression of said DNA sequence(s) results in an altered phenotype; crossing the transgenic fly with a fly containing a mutation in a known or predicted gene; and, screening progeny for flies that display modified expression of the transgenic phenotype as compared to controls. Experimental techniques for performing the steps involved in the screen described above are described, for example, in Cohen et al., (U.S. 20020174446A1), or Benzer et al., (WO200112238A1), herein incorporated by reference.

### DETAILED DESCRIPTION

The present invention discloses transgenic flies that express human Aβ42_{Dutch}, containing a E22Q mutation, either alone or in combination with the Tau protein. The transgenic flies exhibit progressive neurodegeneration which can lead to a variety of altered phenotypes including locomotor phenotypes, behavioral phenotypes (e.g., appetite, mating behavior, and/or life span), and morphological phenotypes (e.g., shape, size, or location of a cell, organ, or appendage; or size, shape, or growth rate of the fly).

As used herein, the term “transgenic fly” refers to a fly whose somatic and germ cells comprise a transgene operatively linked to a promoter, wherein the transgene encodes the human Aβ42_{Dutch}, and wherein the expression of said transgenes in the nervous system results in said Drosophila having a predisposition to, or resulting in, progressive neural degeneration. The term “double transgenic fly” refers to a transgenic fly whose somatic and germ cells comprise at least two transgenes, wherein the transgenes encode the Tau and human Aβ42_{Dutch}. Although the exemplified double transgenic fly is produced by crossing two single transgenic flies, the double transgenic fly of the present invention can be produced using any method known in the art for introducing foreign DNA into an animal. The terms “transgenic fly” and “double transgenic fly” include all developmental stages of the fly, i.e., embryonic, larval, pupal, and adult stages. The development of Drosophila is temperature dependent. The Drosophila egg is about half a millimeter long. It takes about one day after fertilization for the embryo to develop and hatch into a worm-like larva. The larva eats and grows continuously, molting one day, two days, and four days after hatching (first, second and third instars). After two days as a third instar larva, it molts one more time to form an immobile pupa. Over the next four days, the body is completely remodeled to give the adult winged form, which then hatches from the pupal case and is fertile after another day (timing of development is for 25° C.; at 18°, development takes twice as long).

As used herein, “fly” refers to an insect with wings, such as Drosophila. As used herein, the term “Drosophila” refers to any member of the Drosophilidae family, which include without limitation, Drosophila funebris, Drosophila multispina, Drosophila subfunebris, guttiferaspecies group, Drosophila guttifera, Drosophila albomicans, Drosophila annulipes, Drosophila curviceps, Drosophila formosana, Drosophila hypocausta, Drosophila immigrans, Drosophila keplauana, Drosophila kohkoa, Drosophila nasuta, Drosophila neohypocausta, Drosophila niveifrons, Drosophila pallidiftons, Drosophila pulaua, Drosophila quadrilineata, Drosophila siamana, Drosophila sulfurigaster albostrigata, Drosophila sulfurigaster bilimbata, Drosophila sulfurigaster neonasuta, Drosophila Taxon F, Drosophila Taxon I, Drosophila ustulata, Drosophila melanica, Drosophila paramelanica, Drosophila tsigana, Drosophila daruma, Drosophila polychaeta, quinaria species group, Drosophila falleni, Drosophila nigromaculata, Drosophila palustris, Drosophila phalerata, Drosophila subpalustris, Drosophila eohydei, Drosophila hydei, Drosophila lacertosa, Drosophila robusta, Drosophila sordidula, Drosophila repletoides, Drosophila kanekoi, Drosophila virilis, Drosophila maculinatata, Drosophila ponera, Drosophila ananassae, Drosophila atripex, Drosophila bipectinata, Drosophila ercepeae, Drosophila malerkotliana malerkotliana, Drosophila malerkotliana pallens, Drosophila parabipectinata, Drosophila pseudoananassae pseudoananassae, Drosophila pseudoananassae nigrens, Drosophila varians, Drosophila elegans, Drosophila gunungcola, Drosophila eugracilis, Drosophila ficusphila, Drosophila erecta, Drosophila mauritiana, Drosophila melanogaster, Drosophila orena, Drosophila sechellia, Drosophila simulans, Drosophila teissieri, Drosophila yakuba, Drosophila auraria, Drosophila baimaii, Drosophila barbarae, Drosophila biauraria, Drosophila birchii, Drosophila bocki, Drosophila bocqueti, Drosophila burlai, Drosophila constricta (sensu Chen & Okada), Drosophila jambulina, Drosophila khaoyana, Drosophila kikkawai, Drosophila lacteicornis, Drosophila leontia, Drosophila lini, Drosophila mayri, Drosophila parvula, Drosophila pectinifera, Drosophila punjabiensis, Drosophila quadraria, Drosophila rufa, Drosophila seguyi, Drosophila serrata, Drosophila subauraria, Drosophila tani, Drosophila trapezifrons, Drosophila triauraria, Drosophila truncata, Drosophila vulcana, Drosophila watanabei, Drosophila fuyamai, Drosophila biarmipes, Drosophila mimetica, Drosophila pulchrella, Drosophila suzukii, Drosophila unipectinata, Drosophila lutescens, Drosophila paralutea, Drosophila prostipennis, Drosophila takahashii, Drosophila trilutea, Drosophila bifasciata, Drosophila imaii, Drosophila pseudoobscura, Drosophila saltans, Drosophila sturtevanti, Drosophila nebulosa, Drosophila paulistorum, and Drosophila willistoni. In one embodiment, the fly is Drosophila melanogaster.

As used herein, “Aβ42_{Dutch}” is used to refer to a mutant form of the 42-amino acid polypeptide that is produced in nature through the proteolytic cleavage of human amyloid precursor protein (APP) by beta and gamma secretases. Aβ42_{Dutch} differs from wildtype Aβ42 in that it contains a Glu22Gln mutation (SEQ ID NO: 1). Aβ42 is a major component of extracellular amyloid plaque depositions found in neuronal tissue of Alzheimer's disease patients. In the present invention, Aβ42_{Dutch} includes a peptide encoded by a recombinant DNA wherein a nucleotide sequence encoding Aβ42_{Dutch} is operatively linked to an expression control sequence such that the Aβ42_{Dutch} peptide is produced in the absence of cleavage of APP by beta and gamma secretase. It is noted that, because of the degeneracy of the genetic code, different nucleotide sequences can encode the same polypeptide sequence.

As used herein, the term “amyloid plaque depositions” refers to insoluble protein aggregates that are formed extracellularly by the accumulation of amyloid peptides, such as Ap42.

As used herein, the term “signal peptide” refers to a short amino acid sequence, typically less than 20 amino acids in length, which directs proteins to or through the endoplasmic reticulum secretory pathway of Drosophila. “Signal peptides” include, but are not limited to, the Drosophila signal peptides of Dint protein synonymous to “wingless (wg) signal peptide” (SEQ ID NO: 5) and the “Argos (aos) signal peptide” (SEQ ID NO: 6), the Drosophila Appl (SEQ ID NO: 7), presenilin (SEQ ID NO: 8), or windbeutel (SEQ ID NO: 9). Any conventional signal sequence that directs proteins through the endoplasmic reticulum secretory pathway, including variants of the above mentioned signal peptides, can be used in the present invention.

As used herein, an “amino acid linker” refers to a short amino acid sequence from about 2 to 10 amino acids in length that is flanked by two individual peptides.

As used herein, the term “tau protein” refers to the microtubule-associated protein Tau that is involved in microtubule assembly and stabilization. In neuronal tissues of Alzheimer's disease patients, Tau is found in intracellular depositions of neurofibrillary tangles. The human gene that encodes the human Tau protein contains 11 exons, and is described by Andreadis, A. et al., Biochemistry, 31 (43):10626-10633 (1992), herein incorporated by reference. In adult human brain, six tau isoforms are produced from a single gene by alternative mRNA splicing. They differ from each other by the presence or absence of 29- or 58-amino-acid inserts located in the amino-terminal half and 31-amino acid repeat located in the carboxyl-terminal half. Inclusion of the latter, which is encoded by exon 10 of the tau gene, gives rise to the three tau isoforms which each have 4 repeats. As used herein, the term “Tau protein” includes various Tau isoforms produced by alternative mRNA splicing as well as mutant forms of human Tau proteins as described in SEQ ID NO: 4, SEQ ID NO: 10, SEQ ID NO: 11, SEQ ID NO: 12, and SEQ ID 13. In one embodiment, the Tau protein used to generate the double transgenic fly is represented by SEQ ID NO: 3 (amino acid sequence) and SEQ ID NO:4 (nucleotide sequence). In the normal cerebral cortex, there is a slight preponderance of 3 repeat over 4 repeat tau isoforms. These repeats and some adjoining sequences constitute the microtubule-binding domain of tau (Goedert, et al., 1998 Neuron 21, 955-958). In neuronal tissues of Alzheimer's disease patients, Tau is hyperphosphorylated and adopts abnormal and/or pathological conformations detectable using conformational-dependent antibodies, such as MCI and ALZ50 (Jicha G. A., et al., Journal of Neuroscience Research 48:128-132 (1997)). Thus, “Tau protein”, as used herein, includes Tau protein recognized by these conformation specific-antibodies.

The invention further contemplates, as equivalents of these Tau sequences, mutant sequences that retain the biological effect of Tau of forming neurofibrillary tangles. Therefore, “Tau protein”, as used herein, also includes Tau proteins containing mutations and variants. These mutations include but are not limited to: Exon 10+12 “Kumamoto pedigree” (Yasuda et al., (2000) Ann Neurol. 47: 422-9); 1260V (Grover et al., Exp Neurol. 2003 November; 184(1):131-40); G272V (Hutton et al., 1998 Nature 393:702-5; Heutink et al., (1997) Ann Neurol. 41(2):150-9; Spillantini et al., (1996) Acta Neuropathol (Berl). 1996 July;92(1):42-8); N279K (Clark et al., (1998). Proc Natl Acad Sci USA 95: 13103-13107; D'Souza et al., (1999) Proc Natl Acad Sci USA. 96: 5598-5603; Reed et al., (1997) Ann Neurol. 1997 42:564-72; Hasegawa et al., (1999) FEBS Letters 443: 93-96; Hong et al., (1998) Science 282: 1914-1917); delK280 (Rizzu et al., (1999) Am J Hum Genet 64: 414-421; D'Souza et al., (1999) Proc Natl Acad Sci USA. 96: 5598-5603) L284L (D'Souza et al., (1999) Proc Natl Acad Sci USA. 96: 5598-5603); P301L (Hutton et al., 1998 Nature 393:702-5; Heutink et al., (1997) Ann Neurol. 41(2):150-9; Spillantini et al., (1996) Acta Neuropathol (Berl). 1996 July;92(1):42-8; Hasegawa et al., (1998) FEBS Lett. 1998 437(3):207-101; Nacharaju et al., (1999) FEBS Letters 447: 195-199); P301S Bugiani (1999) J Neuropathol Exp Neurol. 58:667-77; Goedert et al., (1999) FEBS Letters 450: 306-311); S305N (Iijima et al., (1999) Neuroreport 10: 497-501; Hasegawa et al., (1998) FEBS Lett. 1998 437(3):207-101; D'Souza et al., (1999) Proc Natl Acad Sci USA. 96: 5598-5603); S305S (Stanford et al., Brain, 123, 880-893, 2000) S305S (Wszolek et al., Brain. 2001 124:1666-70); V337M (Poorkaj et al., (1998) Ann Neurol. 1998 43:815-25; Spillantini et al., (1998) American Journal of Pathology 153: 1359-1363; Sumi et al., (1992) Neurology. 42:120-7; Hasegawa et al., (1998) FEBS Lett. 1998 437(3):207-10); G389R Murrell et al., J Neuropathol Exp Neurol. 1999 December;58(12):1207-26; Pickering-Brown, et al., Ann Neurol. 2000 48(6):859-67); R406W (Hutton et al., 1998 Nature 393:702-5; Reed et al., (1997) Ann Neurol. 1997 42:564-72; Hasegawa et al., (1998) FEBS Lett. 1998 437(3):207-101); 3'Ex10+3, GtoA (Spillantini et al., (1998) American Journal of Pathology 153: 1359-1363; Spillantini et al., (1997) Proc Natl Acad Sci USA. 199794(8):4113-8); 3'Ex10+16 (Baker et al., (1997) Annals of Neurology 42: 794-798; Goedert et al., (1999b) Nature Medicine 5: 454-457; Hutton et al., (1998) Nature 393: 702-705); 3'Ex10+14 (Hutton et al., (1998) Nature 393: 702-705; Lynch et al., (1994) Neurology 44:1878-1884); 3'Ex10+13 (Hutton et al., (1998) Nature 393: 702-705).

Many human Tau gene sequences exist. In adult human brain, six tau isoforms are produced from a single gene by alternative mRNA splicing (Goedert et al., Neuron. 1989 3:519-26). It is noted that, because of the degeneracy of the genetic code, different nucleotide sequences can encode the same polypeptide sequence. The invention further contemplates the use of Tau genes containing sequence polymorphisms (See, for example, Table 1).

TABLE 1
Polymorphisms identified within the human Tau gene. Underlined
polymorphisms are inherited as a part of extended haplotype 2. In case
of exons skipped in the brain mRNA (exon 4a, 6, 8) locations of
polymorphic sites are counted from the first nucleotide of the exon.

| Exon/Intron | Polymorphisms |
|---|---|
| E1 | 5′ UTR−13 a--> g |
| I1 | nt−93 t --> c |
| I2 | nt+18 c --> t |
| I3 | nt+9 a --> g |
| I3 | nt−103 t --> a (very rare on H1) |
| I3 | nt−94a -->t (very rare on H1) |
| E4a | n+232 C --> T (CCG/CTG; P/L) |
| E4a | n+480 G --> A (GAC/AAC; R/N) |
| E4a | n+482 C --> T (GAC/GAT; N/N) |
| E4a | n+493 T --> C (GTA/GCA; V/A) |
| E4a | n316 A --> G (CAA/CGA, Q/Q) |
| I4a | nt−72 t --> c |
| E6 | n+139 C --> T (CAC/TAC H/Y) (very common) |
| E6 | n+157 T --> C (ACT/ACC S/P) |
| I6 | nt+67 a --> g |
| I6 | nt+105 t --> c |
| E7 | P176P (G --> A) |
| E8 | n+5 T --> C (ACT/ACC, T/T) |
| I8 | nt−26 g --> a |
| E9 | A227A (GCA/GCG) |
| E9 | N255N (AAT/AAC) |
| E9 | P270P (CCG/CCA) |
| I9 | nt−47 c --> a (very rare on H1) |
| I9 | Δ238 bp |
| I11 | nt+34 g --> a |
| I11 | nt+90 g --> a |
| I11 | nt+296 c --> t |
| I13 | nt+34 t --> c |

The invention also contemplates the use of Tau proteins or genes from other animals, including but not limited to mice (Lee et al., (1988) Science 239, 285-8), rats (Goedert et al., (1992) Proc. Natl. Acad. Sci. U.S.A. 89 (5), 1983-1987), Bos taurus (Himmler et al., (1989) Mol. Cell. Biol. 9 (4), 1381-1388), Drosophila melanogaster (Heidary & Fortini, (2001) Mech. Dev. 108 (1-2), 171-178) and Xenopus laevis (Olesen et al., (2002) Gene 283 (1-2), 299-309). The Tau genes from other animals may additionally contain mutations equivalent to those previously described. Equivalent positions can be identified by sequence alignment, and equivalent mutations can be introduced by means of site-directed mutagenesis or other means known in the art.

As used herein, the term “neurofibrillary tangles” refers to insoluble twisted fibers that form intracellularly and that are composed mainly of Tau protein.

As used herein, the term “operatively linked” refers to a juxtaposition wherein the components described are in a relationship permitting them to function in their intended manner. An expression control sequence “operatively linked” to a coding sequence is ligated in such a way that expression of the coding sequence is achieved under conditions compatible with the activity of the control sequences.

As used herein, the term “expression control sequence” refers to promoters, enhancer elements, and other nucleic acid sequences that contribute to the regulated expression of a given nucleic acid sequence. The term “promoter” refers to DNA sequences recognized by RNA polymerase during initiation of transcription and can include enhancer elements. As used herein, the term “enhancer element” refers to a cis-acting nucleic acid element, which controls transcription initiation from homologous as well as heterologous promoters independent of distance and orientation. Preferably, an “enhancer element” also controls the tissue and temporal specification of transcription initiation. In particular embodiments, enhancer elements include, but are not limited to, the UAS control element. “UAS” as used herein, refers to an Upstream Activating Sequence recognized and bound by the Gal4 transcriptional activator. The term “UAS control element”, as used herein, refers to a UAS element that is activated by Gal4 transcriptional regulator protein. A “tissue specific” expression control sequence, as used herein, refers to expression control sequences that drive expression in one tissue or a subset of tissues, while being essentially inactive in at least one other tissue. “Essentially inactive” means that the expression of a sequence operatively linked to a tissue specific expression control sequence is less than 5% of the level of expression of that sequence in that tissue where the expression control sequence is most active. Preferably, the level of expression in the tissue is less than 1% of the maximal activity, or there is no detectable expression of the sequence in the tissue. “Tissue specific expression control sequences” include those that are specific for organs such as the eye, wing, notum, brain, as well as tissues of the central and peripheral nervous systems. Examples of tissue specific control sequences include, but are not limited to, the sevenless promoter/enhancer (Bowtell et al., Genes Dev. 2(6):620-34 (1988)); the eyeless promoter/enhancer (Bowtell et al., Proc. Natl. Acad. Sci. U.S.A. 88(15):6853-7 (1991)); gmr/glass responsive promoters/enhancers (Quiring et al., Science 265:785-9 (1994)), and promoters/enhancers derived from any of the rhodopsin genes, that are useful for expression in the eye; enhancers/promoters derived from the dpp or vestigial genes useful for expression in the wing (Staehling-Hampton et al., Cell Growth Differ. 5(6):585-93 (1994)); Kim et al., Nature 382:133-8 (1996)); promoters/enhancers derived from elav (Yao and White, J. Neurochem. 63(1):41-51 (1994)), Appl (Martin-Morris and White, Development 110(1): 185-95 (1990)), and nirvana (Sun et al., Proc. Nat'l Acad. Sci. U.S.A. 96: 10438-43 (1999)) genes useful for expression in the central nervous system; and promoters/enhancers derived from neural specific D42 genes, all of which references are incorporated by reference herein. Other examples of expression control sequences include, but are not limited to the heat shock promoters/enhancers from the hsp70 and hsp83 genes, useful for temperature induced expression; and promoters/enhancers derived from ubiquitously expressed genes, such as tubulin, actin, or Ubiquitin.

As used herein, the term “phenotype” refers to an observable and/or measurable physical, behavioral, or biochemical characteristic of a fly. The term “altered phenotype” as used herein, refers to a phenotype that has changed relative to the phenotype of a wild-type fly. Examples of altered phenotypes include a behavioral phenotype, such as appetite, mating behavior, and/or life span, that has changed by a measurable amount, e.g. by at least 10%, 20%, 30%, 40%, or more preferably 50%, relative to the phenotype of a control fly; or a morphological phenotype that has changed in an observable way, e.g. different growth rate of the fly; or different shape, size, color, or location of an organ or appendage; or different distribution, and/or characteristic of a tissue, as compared to the shape, size, color, location of organs or appendages, or distribution or characteristic of a tissue observed in a control fly. As used herein, “a synergistic altered phenotype” or “synergistic phenotype,” refers to a phenotype wherein a measurable and/or observable physical, behavioral, or biochemical characteristic of a fly is more than the sum of its components.

A “change in phenotype” or “change in altered phenotype,” as used herein, means a measurable and/or observable change in a phenotype relative to the phenotype of a control fly.

As used herein, the “rough eye” phenotype is characterized by irregular ommatidial packing, occasional ommatidial fusions, and missing bristles that can be caused by degeneration of neuronal cells. The eye becomes rough in texture relative to its appearance in wild type flies, and can be easily observed by microscope.

As used herein, the “concave wing” phenotype is characterized by abnormal folding of the fly wing such that wings are bent upwards along their long margins.

As used herein, “locomotor dysfunction” refers to a phenotype where flies have a deficit in motor activity or movement (e.g., at least a 10% difference in a measurable parameter) as compared to control flies. Motor activities include flying, climbing, crawling, and turning. In addition, movement traits where a deficit can be measured include, but are not limited to: i) average total distance traveled over a defined period of time; ii) average distance traveled in one direction over a defined period of time; iii) average speed (average total distance moved per time unit); iv) distance moved in one direction per time unit; v) acceleration (the rate of change of velocity with respect to time; vi) turning; vii) stumbling; viii) spatial position of a fly to a particular defined area or point; ix) path shape of the moving fly; and x) undulations during larval movement; xi) rearing or raising of larval head; and xii) larval tail flick. Examples of movement traits characterized by spatial position include, without limitation: (1) average time spent within a zone of interest (e.g., time spent in bottom, center, or top of a container; number of visits to a defined zone within container); and (2) average distance between a fly and a point of interest (e.g., the center of a zone). Examples of path shape traits include the following: (1) angular velocity (average speed of change in direction of movement); (2) turning (angle between the movement vectors of two consecutive sample intervals); (3) frequency of turning (average amount of turning per unit of time); and (4) stumbling or meander (change in direction of movement relative to the distance). Turning parameters can include smooth movements in turning (as defined by small degrees rotated) and/or rough movements in turning (as defined by large degrees rotated).

As used herein, a “control fly” refers to a larval or adult fly of the same genotype of the transgenic fly as to which it is compared, except that the control fly either i) does not comprise one or both of the transgenes present in the transgenic fly, or ii) has not been administered a candidate agent.

As used herein, the term “candidate agent” refers to a biological or chemical compound that when administered to a transgenic fly has the potential to modify the phenotype of the fly, e.g. partial or complete reversion of the altered phenotype towards the phenotype of a wild type fly. “Agents” as used herein can include any recombinant, modified or natural nucleic acid molecule, library of recombinant, modified or natural nucleic acid molecules, synthetic, modified or natural peptide, library of synthetic, modified or natural peptides; and any organic or inorganic compound, including small molecules, or library of organic or inorganic compounds, including small molecules.

As used herein, the term “small molecule” refers to compounds having a molecular mass of less than 3000 Daltons, preferably less than 2000 or 1500, more preferably less than 1000, and most preferably less than 600 Daltons. Preferably but not necessarily, a small molecule is a compound other than an oligopeptide.

As used herein, a “therapeutic agent” refers to an agent that ameliorates one or more of the symptoms of a neurodegenerative disorder such as Alzheimer's disease in mammals, particularly humans. A therapeutic agent can reduce one or more symptoms of the disorder, delay onset of one or more symptoms, or prevent or cure the disease.

### EXAMPLES

1. Generation of Transgenic Drosophila

A transgenic fly that carries a transgene that encodes the mutant Aβ42_{Dutch} peptide, as well as a double transgenic fly carrying both the Tau protein and the mutant human Aβ42_{Dutch} peptide are disclosed. The transgenic flies provide a model for neurodegenerative disorders such as Alzheimer's disease, which is characterized by an extracellular accumulation of Aβ42_{Dutch} peptide and an intracellular deposition of a hyperphosphorylated form of microtubule-associated protein Tau. The transgenic flies of the present invention can be used to screen for therapeutic agents effective in the treatment of Alzheimer's disease.

A. General

The transgenic flies of the present invention can be generated by any means known to those skilled in the art. Methods for production and analysis of transgenic Drosophila strains are well established and described in Brand et al., Methods in Cell Biology 44:635-654 (1994); Hay et al., Proc. Natl. Acad. Sci. USA 94(10):5195-200 (1997); and in Robert D. B. Drosophila: A Practical Approach, Washington D.C. (1986), herein incorporated by reference in their entireties.

In general, to generate a transgenic fly, a transgene of interest is stably incorporated into a fly genome. Any fly can be used, however a preferred fly of the present invention is a member of the Drosophilidae family. An exemplary fly is Drosophila Melanogaster.

A variety of transformation vectors are useful for the generation of the transgenic flies of the present invention, and include, but are not limited to, vectors that contain transposon sequences, which mediate random integration of transgene into the genome, as well as vectors that use homologous recombination (Rong and Golic, Science 288: 2013-2018 (2000)). A preferred vector of the present invention is pUAST (Brand and Perrimon, Development 118:401-415 (1993)) that contains sequences from the transposable P-element which mediate insertion of a transgene of interest into the fly genome. Another preferred vector is PdL that is able to yield doxycycline-dependent overexpression (Nandis, Bhole and Tower, Genome Biology 4 (R8): 1-14, (2003)).

P-element transposon mediated transformation is a commonly used technology for the generation of transgenic flies and is described in detail in Spradling, P element mediated transformation, In Drosophila: A Practical Approach (ed. D. B. Roberts), pp#175-197, IRL Press, Oxford, UK (1986), herein incorporated by reference. Other transformation vectors based on transposable elements, include for example, the hobo element (Blackman et al., Embo J. 8(1):211-7) (1989)), mariner element (Lidholm et al., Genetics 134(3):859-68 (1993)), the hermes element (O'Brochta et al., Genetics 142(3):907-14 (1996)), Minos (Loukeris et al., Proc. Natl. Acad. Sci. USA 92(21):9485-9 (1995)), or the PiggyBac element (Handler et al., Proc. Natl. Acad. Sci. USA 95(13):7520-5 (1998)). In general, the terminal repeat sequences of the transposon that are required for transposition are incorporated into a transformation vector and arranged such that the terminal repeat sequences flank the transgene of interest. It is preferred that the transformation vector contains a marker gene used to identify transgenic animals. Commonly used, marker genes affect the eye color of Drosophila, such as derivatives of the Drosophila white gene (Pyrrotta V., & C. Brockl, EMBO J. 3(3):563-8 (1984)) or the Drosophila rosy gene (Doyle W. et al., Eur. J. Biochem. 239(3):782-95 (1996)) genes. Any gene that results in a reliable and easily measured phenotypic change in transgenic animals can be used as a marker. Examples of other marker genes used for transformation include the yellow gene (Wittkopp P. et al., Curr Biol. 12(18):1547-56 (2002)) that alters bristle and cuticle pigmentation; theforked gene (McLachlan A., Mol Cell Biol. 6(1):1-6 (1986)) that alters bristle morphology; the Adh+ gene used as a selectable marker for the transformation of Adh-strains (McNabb S. et al., Genetics 143(2):897-911 (1996)); the Ddc+ gene used to transform Ddc^{ts2} mutant strains (Scholnick S. et al., Cell 34(1):37-45(1983)); the lacZ gene of E. coli; the neomycin^{R} gene from the E. coli transposon Tn5; and the green fluorescent protein (GFP; Handler and Harrell, Insect Molecular Biology 8:449-457 (1999)), which can be under the control of different promoter/enhancer elements, e.g. eyes, antenna, wing and leg specific promoter/enhancers, or the poly-ubiquitin promoter/enhancer elements.

Plasmid constructs for introduction of the desired transgene are coinjected into Drosophila embryos having an appropriate genetic background, along with a helper plasmid that expresses the specific transposase needed to mobilize the transgene into the genomic DNA. Animals arising from the injected embryos (G0 adults) are selected, or screened manually, for transgenic mosaic animals based on expression of the marker gene phenotype and are subsequently crossed to generate fully transgenic animals (G1 and subsequent generations) that will stably carry one or more copies of the transgene of interest.

Binary systems are commonly used for the generation of transgenic flies, such as the UAS/GAL4 system. This system is a well-established which employs the UAS upstream regulatory sequence for control of promoters by the yeast GAL4 transcriptional activator protein, as described in Brand and Perrimon, Development 118(2):401-15 (1993)) and Rorth et al, Development 125(6):1049-1057 (1998), herein incorporated by reference in their entireties. In this approach, transgenic Drosophila, termed “target” lines, are generated where the gene of interest (e.g. Aβ42_{Dutch} or TAU)) is operatively linked to an appropriate promoter controlled by UAS. Other transgenic Drosophila strains, termed “driver” lines, are generated where the GAL4 coding region is operatively linked to promoters/enhancers that direct the expression of the GAL4 activator protein in specific tissues, such as the eye, antenna, wing, or nervous system. The gene of interest is not expressed in the “target” lines for lack of a transcriptional activator to “drive” transcription from the promoter joined to the gene of interest. However, when the UAS-target line is crossed with a GAL4 driver line, the gene of interest is induced. The resultant progeny display a specific pattern of expression that is characteristic for the GAL4 line.

The technical simplicity of this approach makes it possible to sample the effects of directed expression of the gene of interest in a wide variety of tissues by generating one transgenic target line with the gene of interest, and crossing that target line with a panel of pre-existing driver lines. Individual GAL4 driver Drosophila strains with specific drivers have been established and are available for use (Brand and Perrimon, Development 118(2):401-15 (1993)). Driver strains include, for example apterous-Gal4 (wings, brain, interneurons), elav-Gal4 (CNS), sevenless-Gal4, eyeless-Gal4, GMR-Gal4 (eyes) and the brain specific 7B-Gal4 driver.

B. Generation of Transgenic Flies

The present invention discloses transgenic flies that have incorporated into their genome a DNA sequence that encodes a mutant human Aβ42_{Dutch} fused to a signal peptide, as well as double transgenic flies which comprise a DNA sequence that encodes the Tau protein as well as a DNA sequence encoding the mutant human Aβ42_{Dutch} fused to a signal peptide.

Generation of transgenic flies containing single transgenes can be performed using any standard means known to those skilled in the art. To generate the double transgenic fly, transgenic Drosophila that express either the Aβ42_{Dutch} or the Tau protein are independently made and then crossed to generate a Drosophila that expresses both proteins.

In a preferred embodiment, transgenic Drosophila are produced using the UAS/GAL4 control system. Briefly, to generate a transgenic fly that expresses Tau, a DNA sequence encoding Tau is cloned into a vector such that the sequence is operatively linked to the GAL4 responsive element UAS. Vectors containing UAS elements are commercially available, such as the pUAST vector (Brand and Perrimon, Development 118:401-415 (1993)), which places the UAS sequence element upstream of the transcribed region. The DNA is cloned using standard methods (Sambrook et al., Molecular Biology: A laboratory Approach, Cold Spring Harbor, N.Y. (1989); Ausubel, et al., Current protocols in Molecular Biology, Greene Publishing, Y, (1995)) and is described in more detail under the Molecular Techniques section of the present application. After cloning the DNA into appropriate vector, such as pUAST, the vector is injected into Drosophila embryos (e.g. yw embryos) by standard procedures (Brand et al., Methods in Cell Biology 44:635-654 (1994)); Hay et al., Proc. Natl. Acad. Sci. USA 94(10):5195-200 (1997) to generate transgenic Drosophila.

When the binary UAS/GAL4 system is used, the transgenic progeny can be crossed with Drosophila driver strains to assess the presence of an altered phenotype. A preferred Drosophila comprises the eye specific driver strain gmr-GAL4, which enables identification and classification of transgenics flies based on the severity of the rough eye phenotype. Expression of Tau in Drosophila eye results in the rough eye phenotype (characterized by an eye with irregular ommatidial packing, occasional ommatidial fusions, and missing bristles), which can be easily observed by microscope. The severity of the rough eye phenotype exhibited by a transgenic line, can be classified as strong, medium, or weak. The weak or mild lines have a rough, disorganized appearance covering the ventral portion of the eye. The medium severity lines show greater roughness over the entire eye, while in strong severity lines the entire eye seems to have lost/fused many of the ommatidia and interommatidial bristles, and the entire eye has a smooth, glossy appearance.

To generate a transgenic fly that expresses the mutant human Aβ42, a DNA sequence encoding human Aβ42_{Dutch} is ligated in frame to a DNA sequence encoding a signal peptide such that the Aβ42_{Dutch} peptide can be exported across cell membranes. The signal sequence is directly linked to the Aβ42_{Dutch} coding sequence or indirectly linked by using a DNA linker sequence, for example of 3, 6, 9, 12, or 15 nucleotides. A signal peptide that directs proteins to or through the endoplasmic reticulum secretory pathway of Drosophila is used. Preferred signal peptides of the present invention are the Argos (aos) signal peptide (SEQ ID NO: 6), the wingless (wg) signal peptide (SEQ ID NO: 5) the Drosophila Appl (SEQ ID NO: 7), presenilin (SEQ ID NO: 8), and windbeutel (SEQ ID NO: 9).

The DNA encoding the mutant Aβ42_{Dutch} peptide is linked to a signal sequence by standard ligation techniques and is then cloned into a vector such that the sequence is operatively linked to the GAL4 responsive element UAS. A preferred transformation vector for the generation of Aβ42_{Dutch} transgenic flies is the pUAST vector (Brand and Perrimon, Development 118:401-415 (1993)). As described for the generation of Tau transgenic flies, the vector is injected into Drosophila embryos (e.g. yw embryos) by standard procedures (Brand et al., Meth. in Cell Biol. 44:635-654 (1994)); Hay et al., Proc. Natl. Acad. Sci. USA 94(10):5195-200 (1997)) and progeny are then selected and crossed based on the phenotype of the selected marker gene. When the binary UAS/GAL4 system is used, the transgenic progeny can be crossed with Drosophila driver strains to assess the presence of an altered phenotype. Preferred Drosophila driver strains are gmr-GAL4 (eye) and elav-GAL4 (CNS).

Without being bound to one particular theory, it is believed that the ectopic overexpression of the Aβ42 and/or Tau sequences described herein leads to neurodegeneration which can have numerous cellular, physiological, behavioral and morphological effects. For example, neurodegeneration in the eye is believed to give rise to the rough eye phenotype; neurodegeneration in the wing (i.e., neuromuscular degeneration) is believed to give rise to morphological wing abnormalities such as the concave wing; and neurodegeneration in the CNS or PNS is believed to give rise to numerous locomotive and behavioral phenotypes. Abberant overexpression of the Aβ42 and/or Tau sequences described herein may be evaluated by screening flies for phenotypic changes which are commensurate with the tissue-specific expression of the sequence as dictated by a particular expression control sequence. For example, were the gmr, sevenless, eyeless, or rhodopsin-derived eye-specific promoter/enhancer is used to direct expression in the eye, a phenotype such as the rough-eye phenotype is expected to be observed. Where an enhancer/promoter derived from the dpp or vestigial genes is used to direct expression in the wing, a phenotype such as the concave wing is expected to be obaerved. Where a promoter/enhancer derived from elav, Appl, or nirvana is used to direct expression in the central nervous system, or a promoter/enhancer derived from neural specific D42 genes is used, neurological, locomotor, and/or behavioral phenotypes can be expected to be obeserved. The converse approach is also contemplated. For example, to assess an eye phenotype (e.g., rough eye phenotype) a gmr-GAL4 driver strain is used in the cross. Ectopic overexpression of mutant Aβ42_{Dutch} in Drosophila eye is believed to disrupt the regular trapezoidal arrangement of the photoreceptor cells of the ommatidia (identical single units, forming the Drosophila compound eye), the severity of which is believed to depend on transgene copy number and expression levels. To evaluate a locomotor phenotype (e.g., climbing assay), an elav (or other neural specific promoter)-Gal4 driver strain is used in the cross. Ectopic overexpression of mutant Aβ42_{Dutch} in Drosophila central nervous system (CNS) is believed to result in locomotor deficiencies, such as impaired movement, climbing and flying. To evaluate a wing phenotype (e.g., concave wing), a dpp- or vestigial-Gal4 driver strain is used in the cross. Ectopic overexpression of mutant Aβ42_{Dutch} in Drosophila wing is believed to result in a concave wing phenotype, evidenced by abnormal folding of the fly wing such that wings are bent upwards along their long margins.

Once the single transgenic flies are produced, the flies can be crossed with each other by mating. Flies are crossed according to conventional methods. When the binary UAS/GAL4 system is used, the fly is crossed with an appropriate driver strain and the altered phenotype assessed, as described above, transgenic flies are classified by assessing phenotypic severity. For example, as disclosed herein, the combination of Tau and mutant Aβ42_{Dutch} transgenes is believed to produce a synergistic effect on the eye.

Expression of Tau and mutant Aβ42_{Dutch} proteins in transgenic flies is confirmed by standard techniques, such as Western blot analysis or by immunostaining of Drosophila tissue cross-sections, both of which are described below.

a. Western Blot Analysis

Western blot analysis is performed by standard methods. Briefly, as means of example, to detect expression of the Aβ42 peptide or Tau by western blot analysis, whole flies, or Drosophila heads (e.g. 80-90 heads) are collected and placed in an eppendorf tube on dry ice containing 100 μl of 2% SDS, 30% sucrose, 0.718 M Bistris, 0.318 M Bicine, with “Complete” protease inhibitors (Boehringer Mannheim), then ground using a mechanical homogenizer. Samples are heated for 5 min at 95° C., spun down for 5 min at 12,000 rpm, and supernatants are transferred into a fresh eppendorf tube. 5% β-mercaptoethanol and 0.01% bromphenol blue are added and samples are boiled prior to loading on a separating gel. Approximately 200 ng of total protein extract is loaded for each sample, on a 15% Tricine/Tris SDS PAGE gel containing 8M Urea. After separating, samples are then transferred to PVDF membranes (BIO-RAD, 162-0174) and the membranes are subsequently boiled in PBS for 3 min. Anti-Tau antibody (e.g. T14 (Zymed) and AT100 (Pierce-Endogen) or anti-β42 antibody (e.g. 6E10 (Senetek PLC Napa, Calif.) are hybridized, generally at a concentration of 1:2000, in 5% non-fat milk, 1×PBS containing 0.1% Tween 20, for 90 min at room temperature. Samples are washed 3 times for 5 min., 15 min. and 15 min. each, in 1×PBS-0.1% Tween-20. Labeled secondary antibody, (for example, anti-mouse-HRP from Amersham Pharmacia Biotech, NA 931) is prepared, typically at a concentration of 1:2000, in 5% non-fat milk, 1×PBS containing 0.1% Tween 20, for 90 min at room temperature. Samples are then washed 3 times for 5 min., 15 min. and 15 min. each, in 1×PBS-0.1% Tween-20. Protein is then detected using the appropriate method. For example, when anti-mouse-HRP is used as the conjugated secondary antibody, ECL (ECL Western Blotting Detection Reagents, Amersham Pharmacia Biotech, # RPN 2209) is used for detection.

b. Cross Sections

As a manner of confirming protein expression in transgenic flies, immunostaining of Drosophila organ cross sections is performed. Such a method is of particular use to confirm the presence of hyperphosphorylated Tau, which is a modified form of the Tau protein that is present in non-diseased tissue. Hyperphosphorylated Tau exhibits altered pathological conformations as compared to Tau protein and is present in diseased tissue from patients with certain neurodegenerative disorders, such as Alzheimer's disease.

Cross sections of Drosophila organs can be made by any conventional cryosectioning, such as the method described in Wolff, Drosophila Protocols, CSHL Press (2000), herein incorporated by reference. Cryosections can then be immunostained for detection of Tau and Aβ42 peptides using methods well known in the art. In a preferred embodiment, the Vectastain ABC Kit (which comprises biotinylated anti-mouse IgG secondary antibody, and avidin/biotin conjugated to the enzyme Horseradish peroxidase H (Vector Laboratories) is used to identify the protein. In other embodiments the secondary antibody is conjugated to a fluorophore. Briefly, cryosections are blocked using normal horse serum, according to the Vectastain ABC Kit protocol. The primary antibody, recognizing the human Aβ42 peptide or Tau, is typically used at a dilution of 1:3000 and incubation with the secondary antibody is done in PBS/1% BSA containing 1-2% normal horse serum, also according to the Vectastain ABC Kit protocol. The procedure for the ABC Kit is followed; incubations with the ABC reagent are done in PBS/0.1% saponin, followed by 4×10 minute washes in PBS/0.1% saponin. Sections are then incubated in 0.5 ml per slide of the Horseradish Peroxidase H substrate solution, 400 ug/ml 3,3′-diaminobenzidene (DAB), 0.006% H 202 in PBS/0.1% saponin, and the reaction is stopped after 3 min. with 0.02% sodium azide in PBS. Sections are rinsed several times in PBS and dehydrated through an ethanol series before mounting in DPX (Fluka).

Exemplary antibodies that can be used to immunostain cross sections include but are not limited to, the monoclonal antibody 6E10 (Senetek PLC Napa, Calif.) that recognizes Aβ42 peptide and anti-Tau antibodies ALZ50 and MCI (Jicha G A, et al., J. of Neurosci. Res. 48:128-132 (1997)).

Alternatively, antibodies for use in the present invention that recognize Aβ42 and Tau can be made using standard protocols known in the art (See, for example, Antibodies: A Laboratory Manual ed. by Harlow and Lane (Cold Spring Harbor Press: 1988)). A mammal, such as a mouse, hamster, or rabbit can be immunized with an immunogenic form of the protein (e.g., a Aβ42 or Tau polypeptide or an antigenic fragment which is capable of eliciting an antibody response). Immunogens for raising antibodies are prepared by mixing the polypeptides (e.g., isolated recombinant polypeptides or synthetic peptides) with adjuvants. Alternatively, Aβ42 or Tau polypeptides or peptides are made as fusion proteins to larger immunogenic proteins. Polypeptides can also be covalently linked to other larger immunogenic proteins, such as keyhole limpet hemocyanin. Alternatively, plasmid or viral vectors encoding Aβ42 or Tau, or a fragment of these proteins, can be used to express the polypeptides and generate an immune response in an animal as described in Costagliola et al., J. Clin. Invest. 105:803-811 (2000), which is incorporated herein by reference. In order to raise antibodies, immunogens are typically administered intradermally, subcutaneously, or intramuscularly to experimental animals such as rabbits, sheep, and mice. In addition to the antibodies discussed above, genetically engineered antibody derivatives can be made, such as single chain antibodies.

The progress of immunization can be monitored by detection of antibody titers in plasma or serum. Standard ELISA, flow cytometry or other immunoassays can also be used with the immunogen as antigen to assess the levels of antibodies. Antibody preparations can be simply serum from an immunized animal, or if desired, polyclonal antibodies can be isolated from the serum by, for example, affinity chromatography using immobilized immunogen.

To produce monoclonal antibodies, antibody-producing splenocytes can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, Nature, 256: 495-497 (1975)), the human B cell hybridoma technique (Kozbar et al., Immunology Today, 4: 72 (1983)), and the EBV-hybridoma technique to produce human monoclonal antibodies (Cole et al., Monoclonal Antibodies and Cancer Therapy, Alan R. Liss, Inc. pp. 77-96(1985)). Hybridoma cells can be screened immunochemically for production of antibodies that are specifically reactive with Aβ42 or Tau peptide, or polypeptide, and monoclonal antibodies isolated from the media of a culture comprising such hybridoma cells.

II. Molecular Techniques

In the present invention, DNA sequences that encode Tau or human Aβ42DUtCh are cloned into transformation vectors suitable for the generation of transgenic flies.

A. Generation of DNA Sequences Encoding Tau or Human Aβ42

DNA sequences encoding Tau and Aβ42_{Dutch} can be obtained from genomic DNA or be generated by synthetic means using methods well known in the art (Sambrook et al., Molecular Biology: A laboratory Approach, Cold Spring Harbor, N.Y. (1989); Ausubel, et al., Current protocols in Molecular Biology, Greene Publishing, Y, (1995)). Briefly, human genomic DNA can be isolated from peripheral blood or mucosal scrapings by phenol extraction, or by extraction with kits such as the QIAamp Tissue kit (Qiagen, Chatsworth, Calif.), Wizard genomic DNA purification kit (Promega, Madison, Wis.), and the ASAP genomic DNA isolation kit (Boehringer Mannheim, Indianapolis, Ind.). DNA sequences encoding Tau and Aβ42_{Dutch} can then be amplified from genomic DNA by polymerase chain reaction (PCR) (Mullis and Faloona Methods Enzymol., 155: 335 (1987)), herein incorporated by reference) and cloned into a suitable recombinant cloning vector.

Alternatively, a cDNA that encodes Tau or human Aβ42_{Dutch} can be amplified from mRNA using RT-PCR and cloned into a suitable recombinant cloning vector. RNA may be prepared by any number of methods known in the art; the choice may depend on the source of the sample. Methods for preparing RNA are described in Davis et al., Basic Methods in Molecular Biology, Elsevier, N.Y., Chapter 11 (1986); Ausubel et al., Current Protocols in Molecular Biology, Chapter 4, John Wiley and Sons, NY (1987); Kawasaki and Wang, PCR Technology, ed. Erlich, Stockton Press N Y (1989); Kawasaki, PCR Protocols: A Guide to Methods and Applications, Innis et al. eds. Academic Press, San Diego (1990); all of which are incorporated herein by reference.

It is preferred, following generation of sequences that encode Tau or Aβ42_{Dutch} by PCR or RT-PCR, that the sequences are cloned into an appropriate sequencing vector in order that the sequence of the cloned fragment can be confirmed by nucleic acid sequencing in both directions.

Suitable recombinant cloning vectors for use in the present invention contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. For example, the origin of replication from the plasmid pBR322 is suitable for most Gram-negative bacteria, the 2 micron plasmid origin is suitable for yeast, and various viral origins (e.g. SV40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector may contain a selection gene also referred to as a selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Since cloning is most conveniently performed in E. coli, an E. coli-selectable marker, for example, the P-lactamase gene that confers resistance to the antibiotic ampicillin, is of use. These can be obtained from E. coli plasmids, such as pBR322 or a pUC plasmid such as pUC18 or pUC 19.

Sequences that encode Tau or human Aβ42_{Dutch} can also be directly cloned into a transformation vector suitable for generation of transgenic Drosophila such as vectors that allow for the insertion of sequences in between transposable elements, or insertion downstream of an UAS element, such as pUAST. Vectors suitable for the generation of transgenic flies preferably contain marker genes such that the transgenic fly can be identified such as, the white gene, the rosy gene, the yellow gene, the forked gene, and others mentioned previously. Suitable vectors can also contain tissue specific control sequences as described earlier, such as, the sevenless promoter/enhancer, the eyeless promoter/enhancer, glass-responsive promoters (gmr)/enhancers useful for expression in the eye; and enhancers/promoters derived from the dpp or vestigial genes useful for expression in the wing.

Sequences that encode Tau or human Aβ42_{Dutch} are ligated into a recombinant vector in such a way that the expression control sequences are operatively linked to the coding sequence.

Herein, DNA sequences that encode Tau or human Aβ42_{Dutch} can be generated through the use of Polymerase chain reaction (PCR), or RT-PCR which uses RNA-directed DNA polymerase (e.g., reverse transcriptase) to synthesize cDNAs which is then used for PCR.

III. Phenotypes and Methods of Detecting Altered Phenotypes

A double transgenic fly according to the invention can exhibit an altered eye phenotype, of progressive neurodegeneration in the eye that leads to measurable morphological changes in the eye (Femandez-Funez et al., Nature 408:101-106 (2000); Steffan et. al, Nature 413:739-743 (2001)). The Drosophila eye is composed of a regular trapezoidal arrangement of seven visible rhabdomeres produced by the photoreceptor neurons of each Drosophila ommatidium. A phenotypic eye mutant according to the invention leads to a progressive loss of rhabdomeres and subsequently a rough-textured eye. A rough textured eye phenotype is easily observed by microscope or video camera. In a screening assay for compounds which alter this phenotype, one may observe slowing of the photoreceptor degeneration and improvement of the rough-eye phenotype (Steffan et. al, Nature 413:739-743 (2001)).

A transgenic or double transgenic fly according to the invention can exhibit an altered wing phenotype, believed to be rooted in neuromuscular degeneration in the wing, leading to measurable morphological changes in the wing structure. A concave wing phenotype may be easily observed by microscope, video camera, or other suitable imaging means.

Neuronal degeneration in the central nervous system will give rise to behavioral deficits, including but not limited to locomotor deficits, that can be assayed and quantitated in both larvae and adult Drosophila. For example, failure of Drosophila adult animals to climb in a standard climbing assay (see, e.g. Ganetzky and Flannagan, J. Exp. Gerontology 13:189-196 (1978); LeBourg and Lints, J. Gerontology 28:59-64 (1992)) is quantifiable, and indicative of the degree to which the animals have a motor deficit and neurodegeneration. Neurodegenerative phenotypes include, but are not limited to, progressive loss of neuromuscular control, e.g. of the wings; progressive degeneration of general coordination; progressive degeneration of locomotion, and progressive loss of appetite. Other aspects of Drosophila behavior that can be assayed include but are not limited to circadian behavioral rhythms, feeding behaviors, inhabituation to external stimuli, and odorant conditioning. All of these phenotypes are measured by one skilled in the art by standard visual observation of the fly.

Another neural degeneration phenotype, is a reduced life span, for example, the Drosophila life span can be reduced by 10-80%, e.g., approximately, 30%, 40%, 50%, 60%, or 70%. Any observable and/or measurable physical or biochemical characteristic of a fly is a phenotype that can be assessed according to the present invention. Transgenic flies can be produced by identifying flies that exhibit an altered phenotype as compared to control (e.g., wild-type flies, or flies in which the transgene is not expressed). Therapeutic agents can be identified by screening for agents, that upon administration, result in a change in an altered phenotype of the transgenic fly as compared to a transgenic fly that has not been administered a candidate agent.

A change in an altered phenotype includes either complete or partial reversion of the phenotype observed. Complete reversion is defined as the absence of the altered phenotype, or as 100% reversion of the phenotype to that phenotype observed in control flies. Partial reversion of an altered phenotype can be 5%, 10%, 20%, preferably 30%, more preferably 50%, and most preferably greater than 50% reversion to that phenotype observed in control flies. Example measurable parameters include, but are not limited to, size and shape of organs, such as the eye; distribution of tissues and organs; behavioral phenotypes (such as, appetite and mating); and locomotor ability, such as can be observed in a climbing assays. For example, in a climbing assay, locomotor ability can be assessed by placing flies in a vial, knocking them to the bottom of the vial, then counting the number of flies that climb past a given mark on the vial during a defined period of time. 100% locomotor activity of control flies is represented by the number of flies that climb past the given mark, while flies with an altered locomotor activity can have 80%, 70%, 60%, 50%, preferably less than 50%, or more preferably less than 30% of the activity observed in a control fly population. Locomotor phenotypes also can be assessed as described in provisional application 60/396,339, Methods for Identifying Biologically Active Agents, herein incorporated by reference. Briefly, locomotor dysfunction phenotypes which may be measured according to the invention include deficits in motor activity or movement (e.g., at least a 10% difference in a measurable parameter) as compared to control flies. Motor activities include flying, climbing, crawling, and turning. In addition, movement traits where a deficit can be measured include, but are not limited to: i) average total distance traveled over a defined period of time; ii) average distance traveled in one direction over a defined period of time; iii) average speed (average total distance moved per time unit); iv) distance moved in one direction per time unit; v) acceleration (the rate of change of velocity with respect to time; vi) turning; vii) stumbling; viii) spatial position of a fly to a particular defined area or point; ix) path shape of the moving fly; and x) undulations during larval movement; xi) rearing or raising of larval head; and xii) larval tail flick. Examples of movement traits characterized by spatial position include, without limitation: (1) average time spent within a zone of interest (e.g., time spent in bottom, center, or top of a container; number of visits to a defined zone within container); and (2) average distance between a fly and a point of interest (e.g., the center of a zone). Examples of path shape traits include the following: (1) angular velocity (average speed of change in direction of movement); (2) turning (angle between the movement vectors of two consecutive sample intervals); (3) frequency of turning (average amount of turning per unit of time); and (4) stumbling or meander (change in direction of movement relative to the distance). Turning parameters can include smooth movements in turning (as defined by small degrees rotated) and/or rough movements in turning (as defined by large degrees rotated). Locomoter defects in a fly may be measured using methods known in the art, or by taking measurements including, but not limited to:
a) total distance (average total distance traveled over a defined period of time);
b) X only distance (average distance traveled in X direction over a defined period of time;
c) Y only distance (average distance traveled in Y direction over a defined period of time);
d) average speed (average total distance moved per time unit);
e) average X-only speed (distance moved in X direction per time unit);
f) average Y-only speed (distance moved in Y direction per time unit);
g) acceleration (the rate of change of velocity with respect to time);
h) turning;
i) stumbling;
j) spatial position of one animal to a particular defined area or point (examples of spatial position traits include (1) average time spent within a zone of interest (e.g., time spent in bottom, center, or top of a container; number of visits to a defined zone within container); (2) average distance between an animal and a point of interest (e.g., the center of a zone); (3) average length of the vector connecting two sample points (e.g., the line distance between two animals or between an animal and a defined point or object; e.g. climbing data); (4) average time the length of the vector connecting the two sample points is less than, greater than, or equal to a user defined parameter; and the like);
m) path shape of the moving animal, i.e., a geometrical shape of the path traveled by the animal (examples of path shape traits include the following: (1) angular velocity (average speed of change in direction of movement); (2) turning (angle between the movement vectors of two consecutive sample intervals); (3) frequency of turning (average amount of turning per unit of time); (4) stumbling or meandering (change in direction of movement relative to the distance); and the like. This is different from stumbling as defined above. Turning parameters may include smooth movements in turning (as defined by small degrees rotated) and/or rough movements in turning (as defined by large degrees rotated).
   Memory Assay

In Drosophila, the best characterized assay for associative learning and memory is an odor-avoidance behavioral task (T. Tully, et al. J. Comp. Physiol. A157, 263-277 (1985), incorporated herein by reference). This classical (Pavlovian) conditioning involves exposing the flies to two odors (the conditioned stimuli, or CS), one at a time, in succession. During one of these odor exposures (the CS+), the flies are simultaneously subjected to electric shock (the unconditioned stimulus, or US), whereas exposure to the other odor (the CS−) lacks this negative reinforcement. Following training, the flies are then placed at a ‘choice point’, where the odors come from opposite directions, and expected to decide which odor to avoid. By convention, learning is defined as the fly's performance when testing occurs immediately after training. A single training trial produces strong learning: a typical response is that >90% of the flies avoid the CS+. Performance of wild-type flies from this single-cycle training decays over a roughly 24-hour period until flies once again distribute evenly between the two odors. Flies can also form long-lasting associative olfactory memories, but normally this requires repetitive training regimens.

IV. Utility of Transgenic Flies

A. Disease Model

The transgenic flies of the invention provide a model for neurodegeneration as is found in human neurological diseases such as Alzheimer's and tauopathies, such as Amyotrophic lateral sclerosis/parkinsonism-dementia complex of Guam Argyrophilic grain dementia, Corticobasal degeneration, Dementia pugilistica, Diffuse neurofibrillary tangles with calcification, Frontotemporal dementia with Parkinsonism linked to chromosome 17 (FTDP-17), Pick's disease, Progressive subcortical gliosis, Progressive supranuclear palsy (PSP), Tangle only dementia, Creutzfeldt-Jakob disease, Down syndrome, Gerstmann-Sträussler-Scheinker disease, Hallervorden-Spatz disease, Myotonic dystrophy, Age-related memory impairment, Alzheimer's disease, Amyotrophic lateral sclerosis, Amyotrophic lateral/parkinsonism-dementia complex of Guam, Auto-immune conditions (eg Guillain-Barre syndrome, Lupus), Biswanger's disease, Brain and spinal tumors (including neurofibromatosis), Cerebral amyloid angiopathies (Journal of Alzheimer's Disease vol. 3, 65-73 (2001)), Cerebral palsy, Chronic fatigue syndrome, Creutzfeldt-Jacob disease (including variant form), Corticobasal degeneration, Conditions due to developmental dysfunction of the CNS parenchyma, Conditions due to developmental dysfunction of the cerebrovasculature, Dementia—multi infarct, Dementia—subcortical, Dementia with Lewy bodies, Dementia of human immunodeficiency virus (HIV), Dementia lacking distinct histology, Dendatorubopallidolusian atrophy, Diseases of the eye, ear and vestibular systems involving neurodegeneration (including macular degeneration and glaucoma), Down's syndrome, Dyskinesias (Paroxysmal) Dystonias, Essential tremor, Fahr's syndrome, Friedrich's ataxia, Fronto-temporal dementia and Parkinsonism linked to chromosome 17 (FTDP-17), Frontotemporal lobar degeneration, Frontal lobe dementia, Hepatic encephalopathy, Hereditary spastic paraplegia, Huntington's disease, Hydrocephalus, Pseudotumor Cerebri and other conditions involving CSF dysfunction, Gaucher's disease, Spinal Muscular Atrophy (Hirayama Disease, Werdnig-Hoffman Disease, Kugelberg-Welander Disease), Korsakoff's syndrome, Machado-Joseph disease, Mild cognitive impairment, Monomelic Amyotrophy, Motor neuron diseases, Multiple system atrophy, Multiple sclerosis and other demyelinating conditions (eg leukodystrophies), Myalgic encephalomyelitis, Myotonic dystrophy, Myoclonus Neurodegeneration induced by chemicals, drugs and toxins, Neurological manifestations of Aids including Aids dementia, Neurological conditions (any) arising from polyglutamine expansions, Neurological/cognitive manifestations and consequences of bacterial and/or virus infections, including but not restricted to enteroviruses, Niemann-Pick disease, Non-Guamanian motor neuron disease with neurofibrillary tangles, Non-ketotic hyperglycinemia, Olivo-ponto cerebellar atrophy, Opthalmic and otic conditions involving neurodegeneration, including macular degeneration and glaucoma, Parkinson's disease, Pick's disease, Polio myelitis including non-paralytic polio, Primary lateral sclerosis, Prion diseases including Creutzfeldt-Jakob disease, kuru, fatal familial insomnia, and Gerstmann-Straussler-Scheinker disease, prion protein cerebral amyloid angiopathy, Postencephalitic Parkinsonism, Post-polio syndrome, Prion protein cerebral amyloid angiopathy, Progressive muscular atrophy, Progressive bulbar palsy, Progressive supranuclear palsy, Restless leg syndrome, Rett syndrome, Sandhoff disease, Spasticity, Spino-bulbar muscular atrophy (Kennedy's disease), Spinocerebellar ataxias, Sporadic fronto-temporal dementias, Striatonigral degeneration, Subacute sclerosing panencephalitis, Sulphite oxidase deficiency, Sydenham's chorea, Tangle only dementia, Tay-Sach's disease, Tourette's syndrome, Transmissable spongiform encephalopathies, Vascular dementia, and Wilson disease.

B. Methods for Identifying Therapeutic Agents

The present invention further provides a method for identifying a therapeutic agent for neurodegenerative disease using the transgenic flies disclosed herein. As used herein, a “therapeutic agent” refers to an agent that ameliorates the symptoms of neurodegenerative disease as determined by a physician. For example, a therapeutic agent can reduce one or more symptoms of neurodegenerative disease, delay onset of one or more symptoms, or prevent, or cure.

To screen for a therapeutic agent effective against a neurodegenerative disorder such as disease, a candidate agent is administered to a transgenic fly. The transgenic fly is then assayed for a change in the phenotype as compared to the phenotype displayed by a control transgenic fly that has not been administered a candidate agent. An observed change in phenotype is indicative of an agent that is useful for the treatment of disease.

A candidate agent can be administered by a variety of means. For example, an agent can be administered by applying the candidate agent to the Drosophila culture media, for example by mixing the agent in Drosophila food, such as a yeast paste that can be added to Drosophila cultures. Alternatively, the candidate agent can be prepared in a 1% sucrose solution, and the solution fed to Drosophila for a specified time, such as 10 hours, 12 hours, 24 hours, 48 hours, or 72 hours. In one embodiment, the candidate agent is microinjected into Drosophila hemolymph, as described in WO 00/37938, published Jun. 29, 2000. Other modes of administration include aerosol delivery, for example, by vaporization of the candidate agent.

The candidate agent can be administered at any stage of Drosophila development including fertilized eggs, embryonic, larval and adult stages. In a preferred embodiment, the candidate agent is administered to an adult fly. More preferably, the candidate agent is administered during a larval stage, for example by adding the agent to the Drosophila culture at the third larval instar stage, which is the main larval stage in which eye development takes place.

The agent can be administered in a single dose or multiple doses. Appropriate concentrations can be determined by one skilled in the art, and will depend upon the biological and chemical properties of the agent, as well as the method of administration. For example, concentrations of candidate agents can range from 0.00011 M to 20 mM when delivered orally or through injection, 0.1 μM to 20 mM, 1 μM-10 mM, or 10 μM to 5 mM.

For efficiency of screening the candidate agents, in addition to screening with individual candidate agents, the candidate agents can be administered as a mixture or population of agents, for example a library of agents. As used herein, a “library” of agents is characterized by a mixture more than 20, 100, 10³, 10⁴, 10⁵, 10⁶, 10⁸, 10¹², or 10¹⁵ individual agents. A “population of agents” can be a library or a smaller population such as, a mixture less than 3, 5, 10, or 20 agents. A population of agents can be administered to the transgenic flies and the flies can be screened for complete or partial reversion of a phenotype exhibited by the transgenic flies. When a population of agents results in a change of the transgenic fly phenotype, individual agents of the population can then be assayed independently to identify the particular agent of interest.

In a preferred embodiment, a high throughput screen of candidate agents is performed in which a large number of agents, at least 50 agents, 100 agents or more are tested individually in parallel on a plurality of fly populations. A fly population contains at least 2, 10, 20, 50, 100, or more adult flies or larvae. In one embodiment, locomotor phenotypes, behavioral phenotypes (e.g. appetite, mating behavior, and/or life span), or morphological phenotypes (e.g., shape size, or location of a cell, or organ, or appendage; or size shape, or growth rate of the fly) are observed by creating a digitized movie of the flies in the population and the movie is analyzed for fly phenotype.

B. Candidate Agents

Agents that are useful in the screening assays of the present inventions include biological or chemical compounds that when administered to a transgenic fly have the potential to modify an altered phenotype, e.g. partial or complete reversion of the phenotype. Agents include any recombinant, modified or natural nucleic acid molecule; library of recombinant, modified or natural nucleic acid molecules; synthetic, modified or natural peptides; library of synthetic, modified or natural peptides; organic or inorganic compounds; or library of organic or inorganic compounds, including small molecules. Agents can also be linked to a common or unique tag, which can facilitate recovery of the therapeutic agent.

Example agent sources include, but are not limited to, random peptide libraries as well as combinatorial chemistry-derived molecular library made of D- and/or L-configuration amino acids; phosphopeptides (including, but not limited to, members of random or partially degenerate, directed phosphopeptide libraries; see, e.g., Songyang et al., Cell 72:767-778 (1993)); antibodies (including, but not limited to, polyclonal, monoclonal, humanized, anti-idiotypic, chimeric or single chain antibodies, and FAb, F(ab′)₂ and FAb expression library fragments, and epitope-binding fragments thereof); and small organic or inorganic molecules.

Many libraries are known in the art that can be used, e.g. chemically synthesized libraries, recombinant libraries (e.g., produced by phage), and in vitro translation-based libraries. Examples of chemically synthesized libraries are described in Fodor et al., Science 251:767-773 (1991); Houghten et al., Nature 354:84-86 (1991); Lam et al., Nature 354:82-84 (1991); Medyuski, Bio/Technology 12:709-710 (1994); Gallop et al., J. Medicinal Chemistry 37(9):1233-¹²⁵I (1994); Ohlmeyer et al., Proc. Natl. Acad. Sci. USA 5 90: 10922-10926 (1993); Erb et al., Proc. Natl. Acad. Sci. USA 91:11422-11426 (1994); Houghten et al., Biotechniques 13:412 (1992); Jayawickreme et al., Proc. Natl. Acad. Sci. USA 91:1614-1618 (1994); Salmon et al., Proc. Natl. Acad. Sci. USA 90:11708-11712 (1993); PCT Publication No. WO 93/20242; and Brenner and Lemer, Proc. Natl. Acad. Sci. USA 89:5381-5383 (1992). By way of examples of nonpeptide libraries, a benzodiazopine library (see e.g., Bunin et al., Proc. Natl. Acad. Sci. USA 91:4708-4712 (1994)) can be adapted for use.

Peptoid libraries (Simon et al., Proc. Natl. Acad. Sci. USA 89:9367-9371 (1992)) can also be used. Another example of a library that can be used, in which the amide functionalities in peptides have been permethylated to generate a chemically transformed combinatorial library, is described by Ostreshet al. Proc. Natl. Acad. Sci. USA 91:11138-11142 (1994). Examples of phage display libraries wherein peptide libraries can be produced are described in Scott & Smith, Science 249:386-390 (1990); Devlin et al., Science, 249:404-406 (1990); Christian et al., J. Mol. Biol. 227:711-718 (1992); Lenska, J. Immunol. Meth. 152:149-157 (1992); Kay et al., Gene 128:59-65 (1993); and PCT Publication No. WO 94/18318 dated Aug. 18, 1994.

Agents that can be tested and identified by methods described herein can include, but are not limited to, compounds obtained from any commercial source, including Aldrich (Milwaukee, Wis. 53233), Sigma Chemical (St. Louis, Mo.), Fluka Chemie AG (Buchs, Switzerland) Fluka Chemical Corp. (Ronkonkoma, N.Y.;), Eastman Chemical Company, Fine Chemicals (Kingsport, Tenn.), Boehringer Mannheim GmbH (Mannheim, 25 Germany), Takasago (Rockleigh, N.J.), SST Corporation (Clifton, N.J.), Ferro (Zachary, L A 70791), Riedel-deHaen Aktiengesellschaft (Seelze, Germany), PPG Industries Inc., Fine Chemicals (Pittsburgh, Pa. 15272). Further any kind of natural products may be screened using the methods described herein, including microbial, fungal, plant or animal extracts.

Furthermore, diversity libraries of test agents, including small molecule test compounds, may be utilized. For example, libraries may be commercially obtained from Specs and BioSpecs B. V. (Rijswijk, The Netherlands), Chembridge Corporation (San Diego, Calif.), Contract Service Company (Dolgoprudoy, Moscow Region, Russia), Comgenex USA Inc. (Princeton, N.J.), Maybridge Chemicals Ltd. (Cornwall PL34 OHW, United Kingdom), and Asinex (Moscow, Russia).

Still further, combinatorial library methods known in the art, can be utilized, including, but not limited to: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other approaches are applicable to peptide, non-peptide oligomer or small molecule libraries of compounds (Lam, Anticancer Drug Des.12: 145 (1997)). Combinatorial libraries of test compounds, including small molecule test compounds, can be utilized, and may, for example, be generated as disclosed in Eichler & Houghten, Mol. Med. Today 1:174-180 (1995); Dolle, Mol. Divers. 2:223-236 (1997); and Lam, Anticancer Drug Des. 12:145-167 (1997).

Examples of methods for the synthesis of molecular libraries can be found in the art, for example in: DeWitt et al., Proc. Natl. Acad. Sci. USA 90:6909 (1993); Erb et al., Proc. Natl. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33:2059 (1994); Carell et al., Angew. Chem. Int. Ed. Engl. 33:2061 (1994); and Gallop et al., 15 J. Med. Chem. 37:1233 (1994).

A library of agents can also be a library of nucleic acid molecules; DNA, RNA, or analogs thereof. For example, a cDNA library can be constructed from mRNA collected from a cell, tissue, organ or organism of interest, or genomic DNA can be treated to produce appropriately sized fragments using restriction endonucleases or methods that randomly fragment genomic DNA. A library containing RNA molecules can be constructed, for example, by collecting RNA from cells or by synthesizing the RNA molecules chemically. Diverse libraries of nucleic acid molecules can be made using solid phase synthesis, which facilitates the production of randomized regions in the molecules. If desired, the randomization can be biased to produce a library of nucleic acid molecules containing particular percentages of one or more nucleotides at a position in the molecule (U.S. Pat. No. 5,270,163).

### EXAMPLES

### Example 1

### Generation of Aβ42_{Dutch} and Aβ42/Tau Transgenic Flies

A transgenic Drosophila melanogaster strain containing a transgene encoding Tau and a transgenic Drosophila melanogaster strain containing a transgene encoding human Aβ₄₂_{Dutch} peptide are generated as described herein. The two transgenic fly strains are then crossed to obtain a double transgenic Drosophila melanogaster strain containing both Tau and human Aβ42_{Dutch} genes.

Transgene Constructs

The UAS/GAL4 system are used to generate both the Aβ42_{Dutch} and Tau transgenic flies. A cDNA encoding the longest human brain Tau isoform is cloned using standard ligation techniques (Sambrook et al., Molecular Biology: A laboratory Approach, Cold Spring Harbor, N.Y. 1989) into vector pUAST (Brand and Perrimon, Development 118:401-415 (1993)) as an EcoRI fragment in order to generate transformation vector, pUAS:_{2N4R}Tauwt. The Tau isoform, which is represented by SEQ ID NO: 4 (nucleic acid sequence), and SEQ ID NO: 3 (amino acid sequence) contains Tau exons 2 and 3 as well as four microtubule-binding repeats.

Two pUAST transformation vectors carrying DNA sequences encoding the Aβ42_{Dutch} peptide (SEQ ID NO: 2) are generated. One vector encodes Aβ42_{Dutch} peptide fused to the (pUAS:wg-Aβ42) and another vector encodes Aβ42_{Dutch} peptide fused to Argos (aos) signal peptide (pUAS:aos-Aβ42). To generate pUAS:wg-Aβ42, a DNA sequence encoding Aβ42Dutch peptide is first fused, in frame, to a synthetic oligonucleotide encoding the wingless (wg) signal peptide using a 4 amino acid linker (SFAM). The resulting DNA sequence is then cloned as an EcoRI fragment into vector pUAST (Brand and Perrimon, Development 118:401-415 (1993).

To generate pUAS:aos-Aβ42, the Argos (aos) signal peptide (SEQ ID NO: 6) is PCR amplified from DNA encoding Argos and ligated in frame, to DNA encoding Aβ42_{Dutch} in the absence of a linker sequence. The DNA encoding Argos (aos) signal peptide fused in frame to Aβ42_{Dutch} is cloned into pUAST (Brand and Perrimon, Development 118:401-415 (1993)) as an EcoRI fragment.

Transgenic Strains

To generate transgenic Drosophila lines expressing either Tau or AP⁴²DUtCh the pUAST constructs described above, either pUAS:aos-Aβ42, or pUAS:2N₄RTauwt are injected into a y¹w¹¹⁸ Drosophila Melanogaster embryos as described in (Rubin and Spradling, Science 218:348-353, 1982).

In the case of pUAS:_{2N4R}Tauwt, 6 transgenic lines are generated and classified by visual inspection, as described herein, as strong, medium, and weak based on the severity of the eye phenotype observed after crossing with a gmr-GAL4 driver strain.

In the case of pUAS:aos-Aβ42_{Dutch}, transgenic lines are generated and are classified as strong, medium, and weak based on the severity of the eye phenotype observed after crossing with a gmr-GAL4 driver strain. Transgenic Drosophila strains of moderate eye phenotype that carry the gmr-GAL4 driver and pUAS:aos-Aβ42_{Dutch} or PUAS:_{2N4R}Tauwt are then crossed to generate a double transgenic Drosophila line that express both Tau and human Aβ42_{Dutch} peptide. Crossing the single transgenic flies of moderate eye phenotype should result in a synergistic eye phenotype classified as strong.

In the case of transformation construct pUAS:wg-Aβ42, transgenic lines are generated by injecting the construct into a y¹w¹¹⁸ Drosophila Melanogaster embryos as described in (Rubin and Spradling, Science 218:348-353, 1982) and screened for the insertion of transgene into genomic DNA by monitoring eye color. The pUAST vector carries the white gene marker. Transgenic Drosophila carrying wg-Aβ42_{Dutch} transgene are then crossed with elav-Gal4 driver strains for expression of the transgene in the central nervous system. If the crosses do not result in a measurable phenotype, the transgene is mobilized for expansion of copy number by crossing Transgenic Drosophila carrying wg-Aβ42_{Dutch} transgene with Drosophila that carry a source of P-element. Progeny from this cross are selected based on a change in eye color. Flies carrying higher copy numbers of wg-Aβ42_{Dutch} transgene are then crossed with elav-Gal4 driver strains and locomotor ability of the crossed flies is tested in climbing assays. Transgenic lines may exhibit a locomotor phenotype and the flies are classified as strong, medium, weak and very weak (28 lines) as compared among themselves and to elav-Gal4 driver control flies.

A double transgenic Drosophila carrying wg-AP⁴²DUtch and Tauwt transgenes is then generated by crossing a Tauwt transgenic Drosophila carrying an elav-Gal4 driver, with an wg-AP⁴²DUtch transgenic Drosophila carrying an elav-Gal4 driver. Locomotor ability is assessed and classified as strong, medium, weak and very weak as compared to elav-Gal4 driver control flies.

Climbing performance as a function of age is determined for populations of flies of various genotypes at 27° C. Climbing assays are performed in duplicate (two groups of 30 individuals of the same age.

Drosophila brain is then cyrosectioned, and horizontal cross sections of elav-GAL4; Tauwt/wg-Aβ42_{Dutch} flies are immunostained with anti-Tau conformation dependent antibodies ALZ50 and MCI. Positive staining of neurons may be observed with both MCI antibody (data not shown) and ALZ50 antibody. The result is expected to show that Tau protein, which is expressed in the brain of Aβ42/Tau double transgenic Drosophila, exhibits protein conformations associated with Alzheimer's disease.

Thioflavin-S staining is also performed on cells and neurites of the transgenic flies, described herein, to assess the presence of amyloid. Amyloids, when stained with Thioflavin-S, fluoresce an apple green color under a fluorescent microscope. The methods for Thioflavin-S staining are well known in the art. All flies are developed at 27° C. Thioflavin-S positive cells are not expected to be observed in flies expressing Tau only. Thioflavin-S positive cells are expected to be observed in flies expressing Aβ42_{Dutch} only. However, the number of Thioflavin-S-positive cells is expected to be much greater in flies expressing both Tau and AP⁴²Dutch.

### Example 2

### Screening for a Therapeutic Agent

1. To screen for a therapeutic agent effective against Alzheimer's disease, candidate agents are administered to a plurality of the Aβ42_{Dutch}/Tau transgenic fly larvae that carry the gmr-GAL4 driver and the transgenes UAS:aos-Aβ42_{Dutch} alone or in combination with UAS:_{2N4R}Tauwt. Candidate agents are microinjected into third instar transgenic Drosophila melanogaster larvae (three to 5 day old larvae). Larvae are injected through the cuticle into the hemolymph with defined amounts of each compound using a hypodermic needle of 20 gm internal diameter. Following injection, the larvae are placed into glass vials for completion of their development. After eclosion, the adult flies are anesthetized with CO₂ and visually inspected utilizing a dissecting microscope to assess for the reversion of the Drosophila eye phenotype as compared to control flies in which a candidate agent was not administered. An observed reversion of the Aβ42_{Dutch}/Tau transgenic fly eye phenotype towards the phenotype displayed by the control gmr-GAL4 driver strain is indicative of an agent that is useful for the treatment of Alzheimer's disease.

2. Screening for Memory Effect

Pavlovian Learning

Flies are trained by exposure to electroshock (12 pulses at 60 V, duration of 1.5 seconds, interval of 5 seconds) paired with one odor (benzaldehyde (BA, 4%) or methylcyclohexanol (MCH, 10°) for 60 seconds) and subsequent exposure to a second odor without electroshock. The odor concentrations are adjusted to assume no preference for flies exposed simultaneously to the two odors before the training. Immediately after training, learning is measured by allowing flies to choose between the two odors used during training. No preference between odors results in zero (no learning) performance index (PI). Avoidance of the odor previously paired with electroshock is expected to produce a 0<PI≦1.00 (see Tully, T. and Quinn, W. G., J. Comp. Physiol. A Sens. Neural. Behav. Physiol., 157:263-277 (1985)).

1. A transgenic fly whose genome comprises a DNA sequence encoding a polypeptide comprising the amyloid-β peptide 42 containing the Dutch mutation of SEQ ID: 1.

2. The transgenic fly of claim 1, wherein said transgenic fly is a transgenic Drosophila.

3. The transgenic fly of claim 1, wherein said DNA sequence is operatively linked to an expression control sequence.

4. The transgenic fly of claim 3, wherein said expression control sequence is a tissue specific expression control sequence.

5. The transgenic fly of claim 1, wherein said DNA sequence is fused to a sequence encoding a signal peptide.

6. The transgenic fly of claim 1, wherein said transgenic fly is in one of an embryonic, larval, pupal, or adult stage.

7. A method for identifying an agent active in neurodegenerative disease, comprising the steps of:
(a) providing a first transgenic fly according to claim 1 with an observable phenotype;
(b) contacting said first transgenic fly with a candidate agent; and
(c) observing a phenotype of said first transgenic fly of step (b) relative to the phenotype of a control fly according to claim 1, wherein an observable difference in the phenotype of said first transgenic fly relative to said control fly is indicative of an agent active in neurodegenerative disease.

8. The method of claim 7, wherein said DNA sequence is operatively linked to an expression control sequence.

9. The method of claim 7, wherein said transgenic fly is transgenic Drosophila.

10. The method of claim 7, wherein said transgenic fly is an adult fly.

11. The method of claim 7, wherein said transgenic fly is in its larval stage.

12. The method of claim 8, wherein said expression control sequence is a tissue specific expression control sequence.

13. The method of claim 8, wherein said expression control sequence comprises a UAS control element.

14. The method of claim 7, wherein said first DNA sequence is fused to a sequence encoding a signal peptide.

15. The method of claim 14, wherein said signal peptide is the wingless (wg) signal peptide.

16. The method of claim 14, wherein said signal peptide is the Argos (aos) signal peptide.

17. The method of claim 7, wherein said observable phenotype is a selected from the group consisting of: rough eye phenotype; concave wing phenotype; behavioral phenotype; and locomotor dysfunction.

18. A method for identifying an agent active in neurodegenerative disease, comprising the steps of:
(a) providing a transgenic fly according to claim 1 and a control wild-type fly;
(b) contacting said first transgenic fly and said control wild-type fly with a candidate agent; and
(c) observing a difference in phenotype between said transgenic fly and said control fly, wherein a difference in phenotype is indicative of an agent active in neurodegenerative disease.

19. The method of claim 18, wherein each of said first and second DNA sequences is operatively linked to an expression control sequence.

20. The method of claim 18, wherein said transgenic fly is transgenic Drosophila.

21. The method of claim 18, wherein said transgenic fly is an adult fly.

22. The method of claim 18, wherein said transgenic fly is in its larval stage.

23. The method of claim 19, wherein said expression control sequence is a tissue specific expression control sequence.

24. The method of claim 19, wherein said expression control sequence comprises a UAS control element.

25. The method of claim 18, wherein said first DNA sequence is fused to a signal peptide.

26. The method of claim 18, wherein said signal peptide is the wingless (wg) signal peptide.

27. The method of claim 18, wherein said signal peptide is the Argos (aos) signal peptide.

28. The method of claim 18 wherein said observable phenotype is selected from the group consisting of: rough eye phenotype; concave wing phenotype; behavioral phenotype; and
locomotor dysfunction.

29. A transgenic fly whose genome comprises a first DNA sequence that encodes a human arnyloidid-β peptide 42 containing the Dutch mutation of SEQ ID: 1, and a second DNA sequence that encodes a Tau protein.

30. The transgenic fly of claim 29, wherein each of said first and second DNA sequences is operatively linked to an expression control sequence.

31. The transgenic fly of claim 29, wherein said transgenic fly is a transgenic Drosophila.

32. The transgenic fly of claim 30, wherein said expression control sequence is a tissue specific expression control sequence.

33. The transgenic fly of claim 29, wherein said DNA sequence is fused to a signal sequence.

34. The transgenic fly of claim 29, wherein said transgenic fly is in one of an embryonic, larval, pupal, or adult stage.

35. A method for identifying an agent active in neurodegenerative disease, comprising the steps of:
(a) providing a first transgenic fly according to claim 29 with an observable phenotype;
(b) contacting said first transgenic fly with a candidate agent; and
(c) observing a phenotype of said first transgenic fly of step (b) relative to the phenotype of a control fly according to claim 18, wherein an observable difference in the phenotype of said first transgenic fly relative to said control fly is indicative of an agent active in neurodegenerative disease.

36. The method of claim 35, wherein said DNA sequence is operatively linked to an expression control sequence.

37. The method of claim 35, wherein said transgenic fly is transgenic Drosophila.

38. The method of claim 35, wherein said transgenic fly is an adult fly.

39. The method of claim 35, wherein said transgenic fly is in its larval stage.

40. The method of claim 36, wherein said expression control sequence is a tissue specific expression control sequence.

41. The method of claim 36, wherein said expression control sequnece comprises a UAS control element.

42. The method of claim 35, wherein said first DNA sequence is fused to a sequence encoding a signal peptide.

43. The method of claim 42, wherein said signal peptide is the wingless (wg) signal peptide.

44. The method of claim 42, wherein said signal peptide is the Argos (aos) signal peptide.

45. The method of claim 35, wherein said observable phenotype is a selected from the group consisting of: rough eye phenotype; concave wing phenotype; behavioral phenotype; and
locomotor dysfunction.

46. A method for identifying an agent active in neurodegenerative disease, comprising the steps of:
(a) providing a transgenic fly according to claim 18 and a control wild-type fly;
(b) contacting said first transgenic fly and said control fly with a candidate agent; and
(c) observing a difference in phenotype between said transgenic fly and said control fly, wherein a difference in phenotype is indicative of an agent active in neurodegenerative disease.

47. The method of claim 46, wherein each of said first and second DNA sequences is operatively linked to an expression control sequence.

48. The method of claim 46, wherein said transgenic fly is transgenic Drosophila.

49. The method of claim 46, wherein said transgenic fly is an adult fly.

50. The method of claim 46, wherein said transgenic fly is in its larval stage.

51. The method of claim 47, wherein said expression control sequence is a tissue specific expression control sequence.

52. The method of claim 47, wherein said expression control sequence comprises a UAS control element.

53. The method of claim 46, wherein said first DNA sequence is fused to a signal peptide.

54. The method of claim 53, wherein said signal peptide is the wingless (wg) signal peptide.

55. The method of claim 53, wherein said signal peptide is the Argos (aos) signal peptide.

56. The method of claim 46 wherein said observable phenotype is selected from the group consisting of: rough eye phenotype; concave wing phenotype; behavioral phenotype; and locomotor dysfunction.
